(19) United States
(12) Patent Application Publication
(10) Pub. No.: US 2004/0097963 A1
(43) Pub. Date: May 20, 2004

(54) Method and apparatus for disintegrating urinary tract stones

(76) Inventor: J. Michael Seddon, Dunn, NC (US)

Correspondence Address: COATS & BENNETT, PLLC, P O BOX 5, RALEIGH, NC, 27602, US

(21) Appl. No.: 10/299,432

(22) Filed: Nov. 19, 2002

(51) Int. Cl. A61B 17/22

(52) U.S. Cl. 606/127

(57) ABSTRACT

A method and apparatus for treating urinary tract stones in the urinary tract uses an adjustable stone retainer to engage and retain a urinary tract stone and a central electrode to apply disintegrating energy to the retained urinary tract stone. A physician inserts a collapsed retainer assembly, including the adjustable stone retainer and the central electrode enclosed in an outer sheath, into the urinary tract. Once the probe is proximate the urinary tract stone, the physician extends the stone retainer relative to the outer sheath and engages and retains the urinary tract stone in the stone retainer. While retaining the urinary tract stone in the stone retainer, the physician applies disintegrating energy with the central electrode to the urinary tract stone until the urinary tract stone disintegrates. Once the treatment is complete, the physician retracts the stone retainer into the outer sheath and removes the collapsed retainer assembly from the urinary tract.

### BACKGROUND OF THE INVENTION

The present invention generally relates to a method and apparatus for disintegrating urinary tract stones, and more particularly to a method and apparatus for capturing and retaining a urinary tract stone while disintegrating the urinary tract stone.

The urinary tract, comprising the kidneys, ureters, bladder, and urethra, functions to remove waste and extra water from the body. Kidneys, bean-shaped organs approximately the size of a fist, sift waste and extra water from the blood stream. The waste and extra water becomes urine, which flows through the ureters and into the bladder, where it is stored. Once the bladder becomes full, the urine exits the body via the urethra.

Normally, the urine contains chemicals to prevent crystalline build up within the kidneys. However, sometimes these chemicals are either ineffective or absent from the urine. In these instances, crystalline build-up may occur within the kidneys, forming urinary tract stones. Often such stones exit the body unnoticed. However, if a stone becomes large, it may have difficulty passing through the ureters or the urethra and cause great pain to the individual and/or possible damage to the urinary tract.

There are several techniques available for treating painful urinary tract stones. Surgery, medication, increased fluid intake, Extracorporeal Shock Wave Lithotripsy (ESWL), Electro-Hydraulic Lithotripsy (EHL), and Holmium laser comprise some common treatment methods. While medication effectively treats some urinary tract stones, it is generally ineffective in treating calcium-based stones, the most common type of urinary tract stone in the United States. When medication and/or increased fluid intake are ineffective, ESWL, EHL, and holmium laser become the preferred treatment options in most cases because of the reduced recovery time as compared to surgery. EHL and holmium laser, particularly effective for treating stones located in the ureter, are commonly used to treat larger stones and/or when ESWL fails.

One traditional treatment option uses a ureteroscope to locate the urinary tract stone. A surgeon passes the ureteroscope, a small thin endoscope, through the urethra and bladder into the ureter. After visualizing the stone, the surgeon may capture the stone with a cage-like device passed into the ureter through the ureteroscope. The physician then pulls the cage-like device containing the stone through the ureter, bladder, and urethra, to remove the stone from the body.

Alternatively, an EHL probe, comprising a two-contact electrode, may be passed into the ureter through the ureteroscope. Disintegrating energy generated by the probe creates a small spark at a high temperature that vaporizes a small bubble of water. The bubble expands, like a shock wave, and fragments the stone. This is a very delicate operation due to the close proximity of the electrode to nearby tissue and ureteroscope. When properly aimed at the stone, repeated application of the disintegrating energy will generally disintegrate most urinary tract stones. However, it is difficult to aim the probe directly at a urinary tract stone and stay clear of the ureter. This is because the instrument channel in the ureteroscope is of necessity towards the periphery of the ureteroscope. As a result, the probe tends to contact the periphery of the urinary tract stone, close to the ureteric wall. Further, the shock wave associated with some disintegrating energies may cause the urinary tract stone(s) to move. As a result, the doctor often ends up “chasing” the stone in the ureter in order to apply the necessary disintegrating energy to the stone.

Therefore, there is a need for a method and apparatus for capturing and retaining the urinary tract stone while directing disintegrating energy towards the stone. There is also a need to direct this energy to the center of the stone, minimizing damage to the stone retaining device.

### SUMMARY OF THE INVENTION

The present invention is directed to a method and apparatus for disintegrating urinary tract stones. In an exemplary embodiment, a stone disintegrator includes a stone retainer for engaging and retaining a urinary tract stone and an associated terminal for directing disintegrating energy towards the retained urinary tract stone.

In exemplary applications, a physician engages and retains the urinary tract stone in a stone retainer. After engaging the urinary tract stone, the physician may retract the stone retainer around the urinary tract stone to further retain the urinary tract stone. Once the urinary tract stone is retained within the stone retainer, the physician directs disintegrating energy towards the retained urinary tract stone with a terminal. The physician may repeatedly direct the disintegrating energy towards the retained urinary tract stone until the stone is disintegrated with no danger of disrupting the retaining device.

### BRIEF DESCRIPTION OF THE DRAWINGS

FIG. 1 illustrates an embodiment of a urinary tract stone disintegrator according to the present invention.

FIG. 2A illustrates a cross section of a urinary tract stone disintegrator with the retainer assembly in a collapsed configuration.

FIG. 2B illustrates a cross section of a urinary tract stone disintegrator with the retainer assembly in an expanded configuration.

FIG. 3 is a schematic view showing the connector and controls for the urinary tract stone disintegrator.

FIG. 4A illustrates a collapsed retainer assembly approaching a urinary tract stone in the urinary tract of a patient.

FIG. 4B illustrates an expanded retainer assembly proximate the urinary tract stone in the urinary tract of the patient.

FIG. 4C illustrates the expanded retainer assembly engaging the urinary tract stone in the urinary tract of the patient.

FIG. 4D illustrates the expanded retainer assembly retaining the urinary tract stone in the urinary tract of the patient.

FIG. 4E illustrates the expanded retainer assembly following disintegration of the urinary tract stone.

FIG. 4F illustrates the collapsed retainer assembly withdrawing from the urinary tract of the patient.

### DETAILED DESCRIPTION OF THE INVENTION

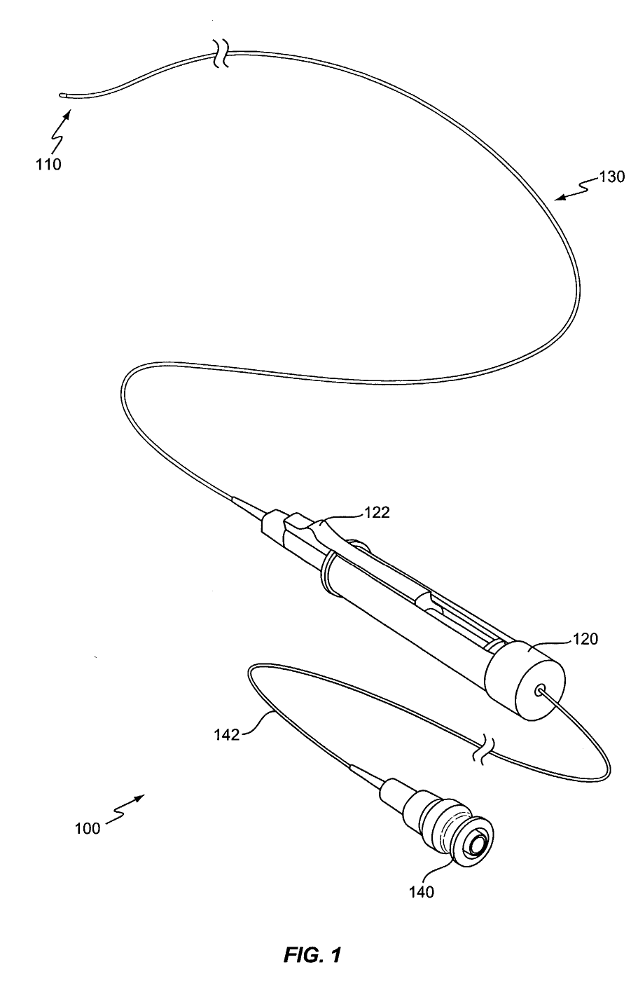

FIG. 1 illustrates an exemplary urinary tract stone disintegrator 100 according to the present invention. Urinary tract stone disintegrator 100 comprises retainer assembly 110 connected to handle 120 via lead cable 130. Urinary tract stone disintegrator 100 also comprises connector 140 coupled to lead cable 130 via handle 120 and connector cable 142.

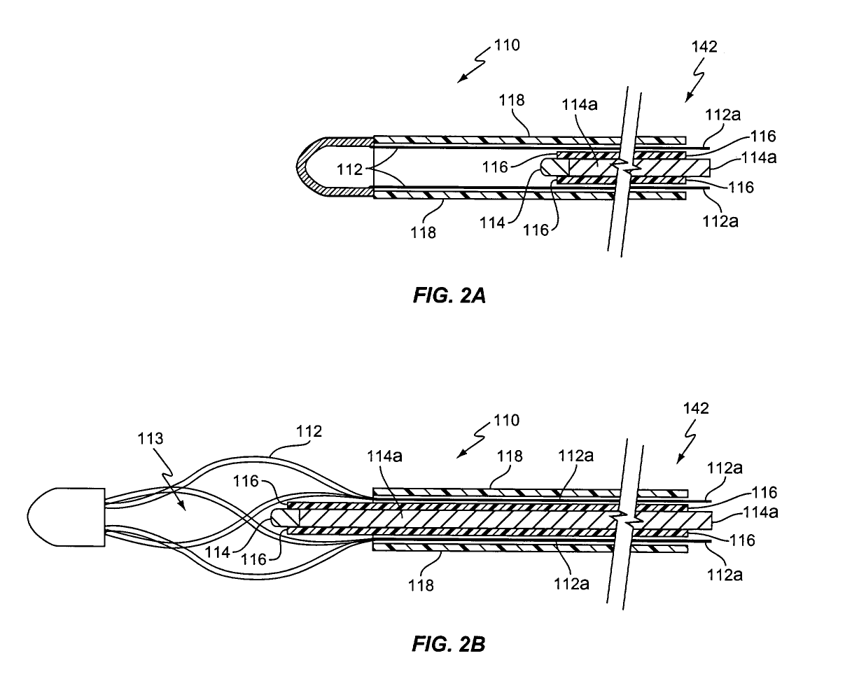

Retainer assembly 110 includes stone retainer 112 and central electrode 114 (FIGS. 2A and 2B). In embodiments described herein, stone retainer 112 is a retaining device that assumes the form of a basket. The present invention may use any retaining device known in the art, i.e., a helical basket, a Segura basket, etc.

Retainer assembly 110 generally assumes one of two basic configurations: a collapsed configuration or an extended configuration. FIG. 2A illustrates a cross-section of retainer assembly 110 in the collapsed configuration. As seen in FIG. 2A, outer sheath 118 encases retainer 112 and central electrode 114 when retainer assembly 110 assumes the collapsed configuration. FIG. 2B illustrates the extended configuration of retainer assembly 110. In the extended configuration, retainer 112 and central electrode 114 extend from outer sheath 118. Once free of outer sheath 118, retainer 112 expands to form a retaining area 113.

Lead cable 130 connects retainer assembly 110 to handle 120, and includes the outer sheath 118 extending from handle 120 to retainer assembly 110. Lead wires connected to retainer assembly 110, are disposed internally to lead cable 130. That is, there is provided an electrical lead wire 114a that extends from the electrode 114 through lead cable 130 and ultimately is electrically connected to the connector 140. Further extending from the retainer 112 through the lead cable 130 back to the handle 120 is a lead wire 112a. Lead wire 112a is in the form of a small sleeve and, like lead wire 114a, is ultimately connected to the connector 140. Extending from the retainer assembly 110 to the handle is an insulator sleeve 116. Insulator sleeve 116 extends around the lead wire 114a and insulates lead wire 112a from lead wire 114a.

Handle 120 provides a control, such as a thumb control 122 movable in a slot, for mechanically controlling the retainer assembly 110. In one embodiment, thumb control 122 is operatively connected to the outer sheath 118. As thumb control 122 is moved back and forth, outer sheath 118 withdraws and advances around retainer assembly 110. Alternatively, thumb control 122 may be operatively connected to retainer assembly 110. In this embodiment, moving thumb control 122 back and forth extends and retracts retainer assembly 110 from and into outer sheath 118. In either embodiment, when retainer assembly 110 extends from outer sheath 118, retainer 112 expands and forms a retaining area 113.

As shown in FIGS. 1-3, a connector 140 connects to retainer assembly 110 via lead cable 130 and a connector cable 142. Connector 140 may be any two-pin connector known in the art. In exemplary embodiments, connector 140 comprises a 2-contact coaxial connector, where an outer contact 146 connects to the retainer lead wire 112a and a center pin 144 connects to the central electrode lead wire 114a. Connector cable 142 encloses the retainer lead wires 112a and the central electrode lead wire 114a in a cable sheath. In addition, the insulator 116 found in lead cable 130 extends through the handle 120 and through the connector cable 142 to insulate the portion of lead wires 112a and 114a extending through connector cable 142. An outer sheath, independent of outer sheath 18 would be provided for connector cable 142.

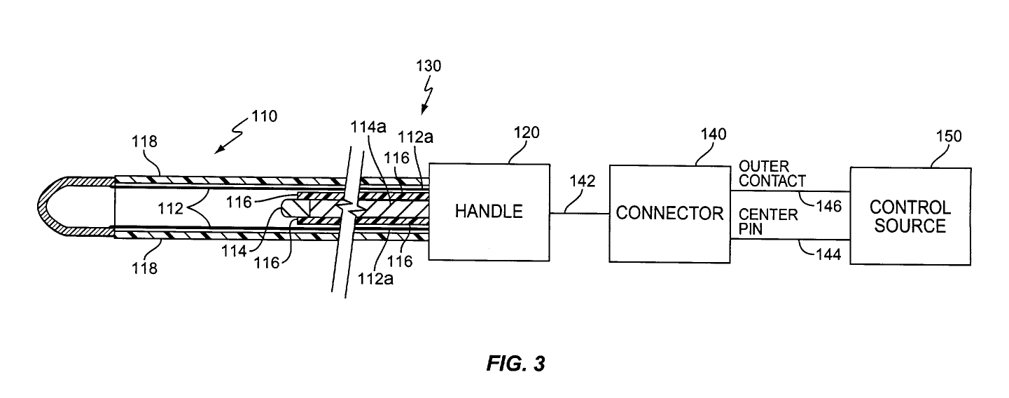

As shown in FIGS. 2-3, when plugged into a control source 150, such as an electro-hydraulic litho machine, connector 140 electrically connects retainer assembly 110 to control source 150. Therefore, it is appreciated that both the central electrode 114 and the retainer 12 would be electrically connected to the control source 150. Control source 150 provides the electrical power and controls necessary to generate disintegrating energy. Details regarding control source 150 are not addressed in the present application because such control devices are well known in the art and are commercially available.

When properly connected to control source 150, activation of control source 150 causes the central electrode 114 to discharge disintegrating energy. Exemplary disintegrating energy includes a high voltage spark that creates an expanding gaseous bubble. As will be understood by those skilled in the art, the power of the disintegrating energy is directly proportional to the distance between retainer 112 and central electrode 114. Therefore, the strength of the disintegrating energy decreases as central electrode 114 moves closer to retainer 112. However, contact between central electrode 114 and retainer 112 creates a short circuit that effectively disables the urinary tract stone disintegrator 100. Also, it is shown that the power of the disintegrating energy is inversely proportional to the amount of central electrode 114 exposed. Therefore, the more central electrode 114 is exposed, the lesser the force. Both of these variables are separate from variability provided by the control source 150.

In exemplary embodiments of the present invention, a fixed, predetermined distance separates central electrode 114 and stone retainer 112. That is, the electrode 114 and stone retainer 112 are secured together. Manipulation of a single thumb control 122 extends and retracts retainer 112 and central electrode 114 with respect to the outer sheath 118. Other embodiments of urinary tract stone disintegrator 100 may include additional controls to move retainer 112 with respect to central electrode 114 and controls with regard to the amount of central electrode 114 exposed.

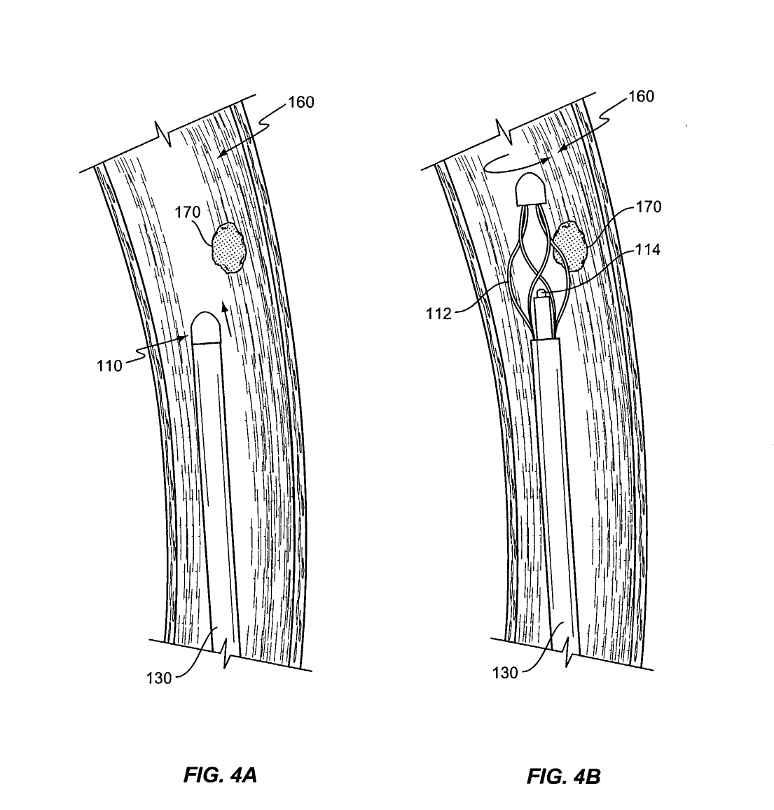

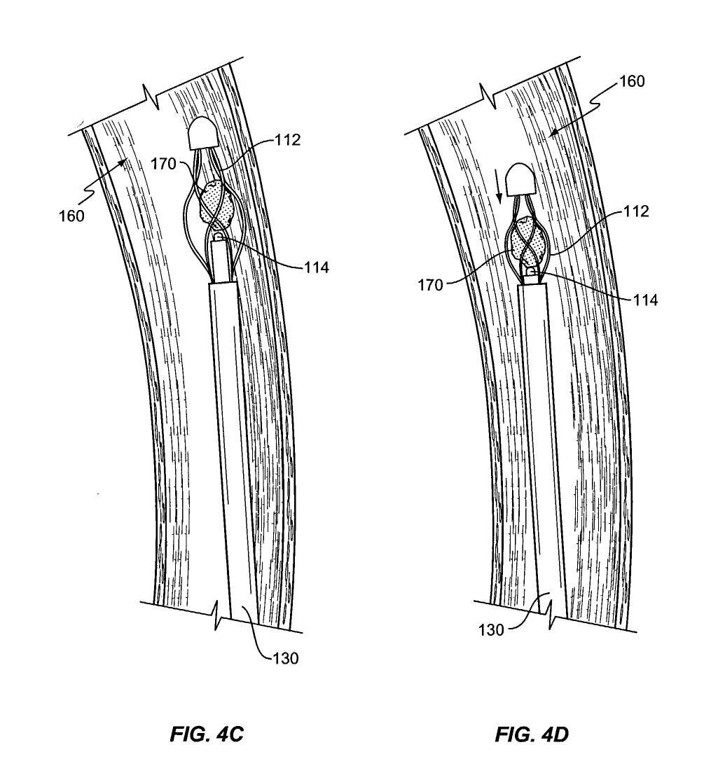

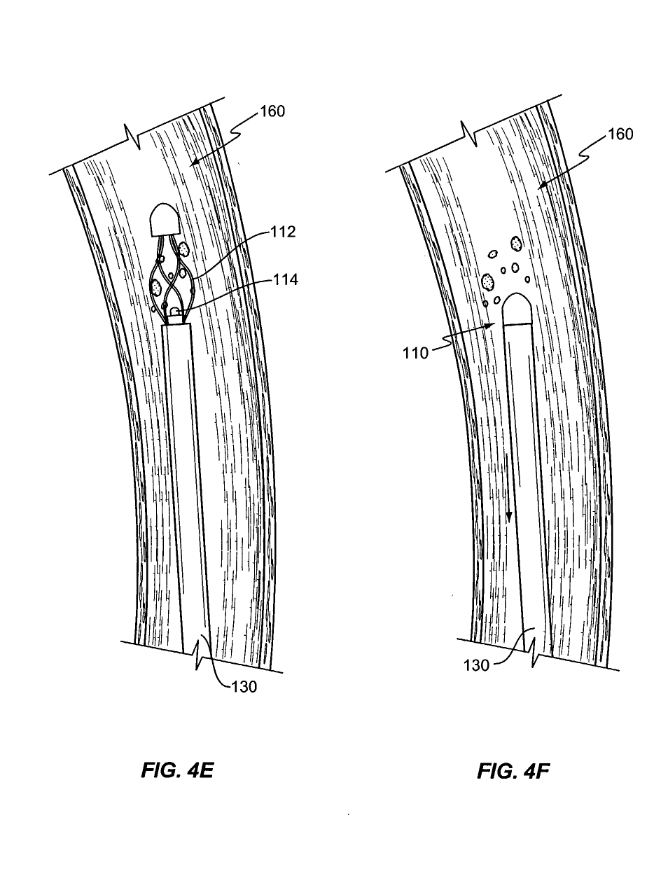

FIGS. 4A-4F illustrate the procedure of using the urinary tract stone disintegrator 100 to disintegrate a urinary tract stone 170 in the urinary tract 160 of a patient. FIGS. 4A-4F are exaggerated for clarity. It will be understood by those skilled in the art that the urinary tract stone disintegrator 100 may be used to treat stones 170 located anywhere in the urinary tract 160, including the kidneys, ureters, and bladder.

Referring to FIG. 4A, a physician inserts a collapsed retainer assembly 110 into the urinary tract 160 of a patient until retainer assembly 110 is adjacent a urinary tract stone 170. The physician may use various techniques, such as ultra-sound images, x-ray images, fiber-optic camera images, etc., to view the location of retainer assembly 110 relative to urinary tract stone 170. Once appropriately positioned, the physician manipulates control 122 (FIG. 1) to extend retainer 112 and central electrode 114 from outer sheath 118. In a preferred embodiment, the physician uses control 122 to manually extend retainer 112 and central electrode 114. However, other controls, such as electronic and/or automatic controls, are also applicable to the present invention, and may be used to extend retainer assembly 110.

In FIGS. 4B-4D, the physician manipulates extended retainer 112, central electrode 114, and/or outer sheath 118 to engage and retain stone 170 in the retainer 112. By manipulating control 122, the physician may retract a portion of retainer 112 and central electrode 114 into the outer sheath 118 to retain stone 170 in retainer 112. In addition, because the power of any disintegrating energy is directly proportional to the distance between retainer 112 and central electrode 114, the physician may decrease the strength of the disintegrating energy by retracting a portion of retainer 112 into the outer sheath 118 to position retainer 112 closer to central electrode 114.

Once urinary tract stone 170 is retained in retainer 112, the physician activates control source 150 (FIG. 3) to apply disintegrating energy to stone 170. In order to amplify the effect of the disintegrating energy, the physician may irrigate the urinary tract 160 with a saline solution before the physician inserts the retainer assembly 110 into the urinary tract 160. In exemplary embodiments, the physician repeatedly applies the disintegrating energy to stone 170 while retracting retainer 112 and central electrode 114 into outer sheath 118, until the physician is satisfied that the urinary tract stone 170 has been disintegrated and can pass safely through the urinary tract 160 (FIG. 4E). The physician will manually activate the control source 150 by the usual methods. Variation in the disintegrating energy may be achieved at the control source 150 by the amount of central electrode 114 exposed within stone retainer 112.

As shown in FIG. 4F, once stone 170 is sufficiently disintegrated, the physician uses control 122 to retract retainer 112 and central electrode 114 into outer sheath 118 such that the retainer assembly 110 once again assumes a collapsed configuration. The physician may then repeat the procedure to treat any other stones 170 present in the urinary tract 160. Once all urinary tract stones have been treated, the physician will remove the collapsed retainer assembly 110 from the urinary tract 160.

The above described method and apparatus provide several benefits over traditional stone treatment options. First, because retainer 112 retains stone 170 proximate central electrode 114, the shock wave resulting from the disintegrating energy no longer moves stone 170 within the urinary tract 160. As a result, the full disintegrating force is applied to the stone 170 and not dissipated elsewhere, making the urinary tract stone disintegrator 100 more efficient than EHL alone. The urinary tract stone disintegrator 100 ensures that the shock wave is initiated at the center of the stone 170; the operator is not required to aim the urinary tract disintegrator 100 at stone 170. Also, stone retainer 112 ensures that the ureteric wall is held as far as possible away from the disintegrating force.

The above benefits could be achieved by simply melding current technologies, i.e., placing the small EHL probe central to a stone-retaining device. However, the present invention provides significant enhancements by making the retaining device part of the electrical circuitry of the EHL probe. First, there is less danger of damaging or disrupting the retaining device. This is because the stone retainer 112 is ground and therefore cannot be damaged by the electrical spark. In addition, as the retaining device approaches the central electrode 114, the size and force of the spark produced by central electrode 114 is decreased. Further, when retainer assembly 110 assumes the collapsed position, retainer 112 and central electrode 114 effectively form a short circuit, effectively disabling retainer assembly 110. Finally, the standard EHL probe deteriorates with repeated sparking because the insulation between the two electrodes fragments with repeated sparking. In the present invention, the space between stone retainer 112 and central electrode 114 represents the corresponding insulation. As a result, no significant deterioration to the device occurs.

Although the apparatus of the present invention has been described in connection with disintegrating urinary tract stones, it is appreciated by those skilled in the art that the present invention can be utilized in other areas of the bodies of humans and animals to disintegrate problematic particles and objects.

The foregoing description and drawings describe and illustrate the present invention in detail. However, the foregoing only describes some embodiments of a urinary tract stone disintegrator. Accordingly, the present invention may be carried out in specific ways other than those set forth herein without departing from the essential characteristics of the invention. The present embodiments are, therefore, to be considered in all respects as illustrative and not restrictive, and all changes coming within the meaning and equivalency range of the appended claims are intended to be embraced therein.

What is claimed is:

1. A method for disintegrating a urinary tract stone comprising
engaging the urinary tract stone and retaining the urinary tract stone in a stone retainer; and
disintegrating the urinary tract stone by directing disintegrating energy towards the urinary tract stone with a terminal while retaining the urinary tract stone in the stone retainer.

2. The method of claim 1 further comprising withdrawing and advancing an outer sheath, adapted to enclose the terminal and the stone retainer, with respect to the terminal and the stone retainer.

3. The method of claim 2 wherein withdrawing the outer sheath extends the stone retainer and terminal from the outer sheath and wherein advancing the outer sheath retracts the stone retainer and terminal into the outer sheath.

4. The method of claim 2 further comprising adjusting the size of the stone retainer by moving the outer sheath with respect to the stone retainer.

5. The method of claim 4 further comprising:
inserting the stone retainer and the terminal enclosed in the outer sheath into a urinary tract of a patient;
extending the stone retainer and terminal from the outer sheath before disintegrating the urinary tract stone;
retracting the stone retainer and terminal into the outer sheath after disintegrating the urinary tract stone; and
removing the stone retainer and terminal enclosed in the outer sheath from the urinary tract of the patient.

6. The method of claim 5 further comprising repeatedly directing disintegrating energy towards the retained urinary tract stone while adjusting the size of the stone retainer.

7. The method of claim 5 wherein extending the stone retainer from the outer sheath causes the stone retainer to expand and spaces the terminal from the urinary tract.

8. The method of claim 7 wherein retracting the stone retainer into the outer sheath encloses the stone retainer in the outer sheath and disables the terminal.

9. The method of claim 1 further comprising repeatedly directing the disintegrating energy towards the urinary tract stone retained within the stone retainer.

10. The method of claim 1 wherein directing the disintegrating energy comprises directing an electric shock towards the urinary tract stone in the stone retainer.

11. The method of claim 1 further comprising visualizing the urinary tract stone.

12. The method of claim 11 wherein visualizing the urinary tract stone comprises using at least one of x-ray, ultra-sound, and camera images.

13. The method of claim 1 further comprising inserting the stone retainer and the terminal into a urinary tract of a patient, and expanding a portion of the stone retainer against the urinary tract such that the terminal is spaced away from the urinary tract.

14. The method of claim 1 further comprising controlling amount of the disintegrating energy by controlling how much of the terminal is exposed.

15. A method for disintegrating a urinary tract stone in a patient comprising:
inserting a terminal and a stone retainer enclosed in an outer sheath into a urinary tract of the patient;
extending the stone retainer and terminal from the outer sheath;
engaging and retaining the urinary tract stone with the stone retainer; and
disintegrating the urinary tract stone by directing disintegrating energy with the terminal towards the urinary tract stone while the urinary tract stone is retained by the stone retainer.

16. The method of claim 15 further comprising adjusting the size of the stone retainer by moving the outer sheath with respect to the stone retainer while repeatedly directing disintegrating energy to the urinary tract stone retained in the stone retainer.

17. The method of claim 15 wherein extending the stone retainer and terminal from the outer sheath comprises withdrawing the outer sheath from the stone retainer and terminal, and wherein retracting the stone retainer and terminal comprises advancing the outer sheath to enclose the stone retainer and terminal.

18. The method of claim 15 further comprising:
retracting the stone retainer and terminal into the outer sheath after disintegrating the urinary tract stone; and
removing the stone retainer and terminal from the urinary tract of the patient.

19. The method of claim 15 wherein directing the disintegrating energy towards the urinary tract stone with the terminal comprises directing an electric shock with an electrode.

20. The method of claim 15 further comprising visualizing the urinary tract stone.

21. The method of claim 20 wherein visualizing the urinary tract stone comprises using at least one of x-ray, ultra-sound, and camera images.

22. A method of breaking up undesirable particles or objects in the body of a human or animal, comprising:
inserting a retainer assembly, comprising a retainer and a terminal, into the body;
engaging the particle or object within the body with the retainer and retaining the particle or object in the retainer; and
disintegrating the particle or object within the body by directing disintergrating energy from the terminal to the particle or object while the particle or object is retained within the retainer.

23. The method of claim 22 wherein the retainer is adapted to assume an open configuration for holding the particle or object, and wherein when the retainer assumes the open configuration the terminal at least partially projects into the open retainer.

24. An apparatus for disintegrating a urinary tract stone comprising:
a stone retainer for engaging and retaining the urinary tract stone; and
an associated terminal for directing disintegrating energy towards the urinary tract stone retained in the stone retainer.

25. The apparatus of claim 24 wherein the stone retainer retains the urinary tract stone proximate the terminal.

26. The apparatus of claim 24 wherein the disintegrating energy comprises an electrical shock.

27. The apparatus of claim 24 further comprising a controller for adjusting an outer sheath with respect to the stone retainer and terminal.

28. The apparatus of claim 24 wherein the stone retainer comprises a basket.

29. The apparatus of claim 28 wherein the basket comprises at least one of a helical basket or a Segura basket.

30. The apparatus of claim 24 wherein the stone retainer includes an expandable retaining section comprising opposing first and second retainer ends with at least one retainer lead extending from the second retainer end, and wherein the terminal includes opposing first and second terminal ends with a terminal lead extending from the second terminal end, and further wherein the first and second terminal ends are located adjacent the second retainer end.

31. The apparatus of claim 30 wherein the terminal lead is disposed in coaxial relationship with the at least one retainer lead, and further wherein the terminal lead and the at least one retainer lead are spaced apart by an insulator.

32. The apparatus of claim 31 further comprising a connector for connecting the terminal and the stone retainer to a control source, wherein the at least one retainer lead electrically connects to a first contact of the connector and the terminal lead electrically connects to a second contact of the connector.

33. The apparatus of claim 32 wherein the first contact of the connector comprises an outer contact of a coaxial connector and wherein the second contact of the connector comprises a pin contact of the coaxial connector.

34. An apparatus for disintegrating a urinary tract stone comprising:
a terminal for directing disintegrating energy towards the urinary tract stone;
an associated stone retainer disposed adjacent to the terminal for engaging the urinary tract stone and retaining the urinary tract stone proximate the terminal while the terminal applies disintegrating energy to the urinary tract stone;
an outer sheath for encasing the stone retainer and the terminal; and
a controller for generating relative movement between the outer sheath and the stone retainer and terminal.

35. The apparatus of claim 34 wherein the disintegrating energy comprises an electrical shock.

36. The apparatus of claim 34 wherein the stone retainer comprises a basket.

37. The apparatus of claim 36 wherein the basket comprises at least one of a helical basket or a Segura basket.

38. The apparatus of claim 34 wherein the controller comprises a manual controller.
